(19) United States
(12) Patent Application Publication
(10) Pub. No.: US 2016/0117452 A1
(43) Pub. Date: Apr. 28, 2016

(54) SYSTEMS AND METHODS FOR PRESCRIPTIONS FOR NONINVASIVE ELECTRICAL BRAIN STIMULATION

(71) Applicant: BRAINIQUE AG, Stans (CH)

(72) Inventors: Raymond Watt, San Diego, CA (US); Jaen Frederik Labuschagne, Cape Town (ZA); Michael J. Slater, Alpine, CA (US)

(73) Assignee: Brainique AG, Stans (CH)

(21) Appl. No.: 14/896,202

(22) PCT Filed: Jun. 9, 2014
(86) PCT No.: PCT/EP2014/061946

§ 371 Date: Dec. 4, 2015

Related U.S. Application Data
(60) Provisional application No. 61/832,727, filed on Jun. 7, 2013.

(51) Int. Cl. G06F 19/00; A61N 1/372; A61N 1/36

(52) U.S. Cl. CPC G06F 19/323; A61N 1/36025; A61N 1/37252; G06F 19/325; G06F 19/3481

(57) ABSTRACT

A computer-implemented method for managing a noninvasive electrical brain stimulation “NIEBS” prescription is provided, the method comprising: receiving data for a prescription for a patient associated with a patient ID at a computer system, wherein said prescription is created by a healthcare professional after examining said patient; validating said healthcare professional and said data for said prescription at said computer system; maintaining a database or library of NIEBS data programs for NIEBS treatments at said computer system; and authorizing a specific NIEBS data program for said patient at said computer system based on said prescription.

### TECHNICAL FIELD

This application relates to systems and methods for prescriptions for noninvasive electrical brain stimulation.

### BACKGROUND

Noninvasive Electrical Brain Stimulation (herein referred to as NIEBS) applies gentle micro-current pulses to the brain using electrodes. It is widely accepted that NIEBS stimulates the brain to manufacture neurotransmitters. Noninvasive electrical brain stimulation has also been proposed for treatment of various medical conditions.

The signals operate to normalize the electrical output of the brain. NIEBS has thus been used/tested to treat substance dependence, depression and anxiety. It has been noted in at least some instances that NIEBS has equal or greater efficacy for the treatment of depression when compared to antidepressant medications, with fewer side effects.

The mechanism by which NIEBS produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

### SUMMARY

According to a first aspect, there is provided a computer-implemented method for managing a noninvasive electrical brain stimulation “NIEBS” prescription, comprising: receiving data for a prescription for a patient associated with a patient ID at a computer system, wherein said prescription is created by a healthcare professional after examining said patient; validating said healthcare professional and said data for said prescription at said computer system; maintaining a database or library of NIEBS data programs for NIEBS treatments at said computer system; and authorizing a specific NIEBS data program for said patient at said computer system based on said prescription.

By receiving data for a prescription for a patient and maintaining a database of NIEBS data programs for NIEBS treatments, it is possible to centralize the storage of NIEBS prescription data in a simple and efficient manner at a computer system so as to enable a user to access NIEBS data from the computer system. By authorizing a data program for a patient, it is possible to allow a user to securely access a NIEBS treatment that has been prescribed to that patient.

According to a second aspect, there is provided a computer-implemented method for delivering a noninvasive electrical brain stimulation “NIEBS” prescription, comprising: sending a request from a device to a server for a NIEBS prescription; receiving said NIEBS prescription from said server, wherein a patient identity associated with said prescription is validated by said server.

By receiving a NIEBS prescription from a server it is possible for a user to securely receive a validated prescription from a server which is appropriate to the user. Accordingly, a more flexible and efficient way of effecting NIEBS treatment is provided.

According to a third aspect, there is provided a computer-implemented method for managing a noninvasive electrical brain stimulation “NIEBS” prescription, comprising: sending a request from a device associated with a patient NIEBS device to a server for a NIEBS prescription; receiving said NIEBS prescription from said server at said device, wherein a patient identity associated with said prescription is validated by said server; and transferring said prescription from said device to said patient NIEBS device.

By receiving a NIEBS prescription at a device and transferring the prescription from that device to a patient NIEBS device, it is possible to centrally access a NIEBS prescription from a server using a device that is able to communication with the server using a communication mechanism enabled on both the server and the device. It is then possible to transfer the prescription to the patient NIEBS device using a separate communication mechanism. It is not necessary for the patient NIEBS device to directly communicate with the server. a more flexible and efficient way of effecting NIEBS treatment is provided

According to a fourth aspect, there is provided a computer-implemented method for delivering a noninvasive electrical brain stimulation “NIEBS” prescription, comprising: receiving a request from a patient device at a server for a NIEBS prescription; validating a patient identity associated with said prescription; upon validation, sending said NIEBS prescription from said server to said patient device to be used by a patient associated with said patient identity for NIEBS treatment.

By receiving a validated NIEBS prescription at a device to be used by a patient for NIEBS treatment, it is possible for the patient to receive the most recently updated prescription from the server in a centralized manner which is simple and efficient.

According to a fifth aspect, there is provided a computer-implemented method for delivering a specific noninvasive electrical brain stimulation “NIEBS” signal formulation for a patient, comprising: providing a prescription for a specific NIEBS signal formulation by a medical professional; forwarding said prescription to a server operated by a service provider of NIEBS services; providing a payment verification mechanism for verifying payment for said prescribed services; and performing authorization for delivery of said NIEBS signal formulation in a computer-readable format by download from the server.

By providing an authorized NIEBS signal formulation in a computer-readable format for download from a server, it is possible for the user device to simply and efficiently access the NIEBS signal formulation which can then be used for NIEBS treatment in a simple and efficient manner by a NIEBS device.

According to a sixth aspect, there is provided an apparatus for delivering a specific NIEBS signal formulation for a patient, comprising: a prescription providing mechanism for providing a prescription for a specific NIEBS signal formulation created by a medical professional; a delivery mechanism for forwarding said prescription to a server operated by a service provider of NIEBS services; a payment verification mechanism for verifying payment for said prescribed services; and an authorization mechanism for providing authorization for delivery of said NIEBS signal formulation in a computer-readable format by download from the server.

By providing an apparatus for delivering a specific NIEBS signal formulation for a patient, it is possible to allow a user to download a specific NIEBS signal formulation for use in a NIEBS treatment in a centralized manner from a simple and efficient manner which enables the user to access the most recently prescribed NIEBS signal formulation. Moreover, the NIEBS signal formulation is provided in a secure manner since authorization and payment verification is provided.

According to a seventh aspect, there is provided a method for managing a NIEBS prescription, comprising: receiving data for a prescription for a patient associated with a patient ID at a third party service provider computer system, wherein said prescription is created by a healthcare professional after examining said patient; validating said healthcare professional and said data for said prescription at said third party service provider computer system; maintaining a database or library of NIEBS data programs for NIEBS treatments at said third party service provider computer system; and authorizing a specific NIEBS data program for said patient at said third party service provider computer system based on said prescription.

By receiving data for a prescription for a patient and maintaining a database of NIEBS data programs for NIEBS treatments, it is possible to centralize the storage of NIEBS prescription data in a simple and efficient manner at a computer system so as to enable a user to access NIEBS data from the computer system. By authorizing a data program for a patient, it is possible to allow a user to securely access a NIEBS treatment that has been prescribed to that patient.

According to an eighth aspect, there is provided a method for delivering a NIEBS prescription, comprising: sending a request from a patient NIEBS device to a server for a NIEBS prescription; receiving said NIEBS prescription from said server, wherein a patient identity associated with said prescription is validated by said server.

By receiving a NIEBS prescription from a server it is possible for a user to securely receive a validated prescription from a server which is appropriate to the user. Accordingly, the NIEBS treatment is more accurately provided to the user. In addition, the prescription is provided in a centralized manner that is simple and efficient.

According to a ninth aspect, there is provided a method for managing a NIEBS prescription, comprising: sending a request from a second device associated with a patient NIEBS device to a server for a NIEBS prescription; receiving said NIEBS prescription from said server at said second device, wherein a patient identity associated with said prescription is validated by said server; and transferring said prescription from said second device to said patient NIEBS device.

By receiving a NIEBS prescription at a device and transferring the prescription from that device to a patient NIEBS device, it is possible to centrally access a NIEBS prescription from a server using a device that is able to communication with the server using a communication mechanism enabled on both the server and the device. It is then possible to transfer the prescription to the patient NIEBS device using a separate communication mechanism. Accordingly, it is not necessary for the patient NIEBS device to directly communicate with the server.

According to a tenth aspect, there is provided a method for delivering a NIEBS prescription, comprising: receiving a request from a patient device at a server for a NIEBS prescription; validating a patient identity associated with said prescription; upon validation, sending said NIEBS prescription from said server to said patient device to be used by a patient associated with said patient identity for NIEBS treatment.

By sending a validated NIEBS prescription at a device to be used by a patient for NIEBS treatment, it is possible for the patient to receive the most recently updated prescription from the server in a centralized manner which is simple and efficient.

According to an eleventh aspect, there is provided a method for delivering a specific NIEBS signal formulation for a patient, comprising: providing a prescription for a specific NIEBS signal formulation by a medical professional; providing a delivery mechanism for forwarding said prescription to a server operated by a service provider of NIEBS services; providing a payment verification mechanism for said prescribed services; and providing authorization for delivery of said NIEBS signal formulation in a computer-readable format by download from the server.

By providing an authorized NIEBS signal formulation for download from a server, it is possible for the user device to simply and efficiently access the NIEBS signal formulation which can then be used for NIEBS treatment in a simple and efficient manner by a NIEBS device.

According to a twelfth apparatus for delivering a specific NIEBS signal formulation for a patient, comprising: a prescription for a specific NIEBS signal formulation created by a medical professional; a delivery mechanism for forwarding said prescription to a server operated by a service provider of NIEBS services; a payment verification mechanism for said prescribed services; and authorization for delivery of said NIEBS signal formulation in a computer-readable format by download from the server.

By providing an apparatus for delivering a specific NIEBS signal formulation for a patient, it is possible to allow a user to download a specific NIEBS signal formulation for use in a NIEBS treatment in a centralized manner from a simple and efficient manner which enables the user to access the most recently prescribed NIEBS signal formulation. Moreover, the NIEBS signal formulation is provided in a secure manner since authorization and payment verification is provided.

### BRIEF DESCRIPTION OF THE DRAWINGS

FIG. 1A illustrates a block diagram of a flowchart for a therapy prescription delivered to a dispensing agency in accordance with embodiments of the present technology.

FIG. 1B illustrates a block diagram of a flowchart for a patient to obtain a NIEBS therapy program via download in accordance with embodiments of the present technology.

FIG. 1C illustrates a block diagram of device for use with a NIEBS prescription in accordance with embodiments of the present technology.

FIG. 1D illustrates a block diagram of device for use with a NIEBS prescription in accordance with embodiments of the present technology.

FIG. 2 illustrates block diagrams of example waveforms for use in a noninvasive electrical brain stimulation system in accordance with embodiments of the present technology.

The drawings referred to in this description of embodiments should be understood as not being drawn to scale except if specifically noted.

### DETAILED DESCRIPTION

Reference will now be made in detail to embodiments of the present technology, examples of which are illustrated in the accompanying drawings. While the technology will be described in conjunction with various embodiment(s), it will be understood that they are not intended to limit the present technology to these embodiments. On the contrary, the present technology is intended to cover alternatives, modifications and equivalents, which may be included within the scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

### Overview of Systems and Methods for Prescriptions for Noninvasive Electrical Brain Stimulation

Embodiments of the present technology are for systems and methods for prescriptions for noninvasive electrical brain stimulation. The description and claims herein specifically describe noninvasive electrical brain stimulation (NIEBS). However, the present technology applies generally to electrotherapy and electro medicine in its many forms. Therefore, the descriptions and claims related to NIEBS may be extended to include electrotherapy in general. Types of electrotherapy may be for, but are not limited to, electro neurostimulation, electro neuromodulation, neuromdulation, brain stimulation, electro medicine, bone growth, muscle stimulation, pain management, etc.

For example, neurostimulation involves modulation of the nervous system and electrically activate neurons in the body. The activation of neural elements in a part of the nervous system can be effectively facilitated by stimulation. Micro-electrodes are utilized to interface with excitable tissue in order to either restore recording experiences to the implant recipient or control an effector organ. Additionally, neuromodulation is the physiological process by which a given neuron uses several different neurotransmitters to regulate diverse populations of central nervous system neurons. This is in contrast to classical synaptic transmission, in which one presynaptic neuron directly influences a single postsynaptic partner. Neuromodulators secreted by a small group of neurons diffuse through large areas of the nervous system, affecting multiple neurons. Examples of neuromodulators include dopamine, serotonin, acetylcholine, histamine and others.

NIEBS is a treatment that applies pulses to the brain across the head of the patient using electrodes. There are many types of NIEBS such as transcranial direct current stimulation (tDCS) which is a form of neuro-stimulation which uses constant, low current delivered directly to the brain area of interest via small electrodes. There are different types of tDCS: anodal, and cathodal. The anodal stimulation is positive (V+) stimulation that increases the neuronal excitability of the area being stimulated. Cathodal (V−) stimulation decreases the neuronal excitability of the area being stimulated. Cathodal stimulation can treat psychological disorders that are caused by the hyper-activity of an area of the brain.

Another form of NIEBS is transcranial alternating current stimulation (tACS) which is a noninvasive means by which alternating currents applied through the skull over the occipital cortex of the brain entrains in a frequency-specific fashion the neural oscillations of the underlying brain. Another class of NIEBS is transcranial pulsed current stimulation (tPCS).

tPCS is a noninvasive method that employs a waveform for use in NIEBS. A tPCS generator is a self-powered device that implements either a fixed tPCS therapy program with preset parameters, or a programmable device that can receive a tPCS therapy program based on treatment options determined by a healthcare professional to be of use to a person with a specific condition. tPCS may also employ a chaotic system that varies many of the pulse characteristics in a random, non-repetitive process.

The present technology is not limited to one form of NIEBS. Therefore, as used herein, NIEBS may refer to many varieties of NIEBS including, but not limited to, transcranial direct current stimulation (tDCS), transcranial alternating current stimulation (tACS), tPCS, and any other neuro-stimulation type protocols such as random noise stimulation and chaotic noise stimulation.

NIEBS involves brain stimulation by low current low voltage that may use alternating square waves or other waves. The effect is to improve the brain's “plasticity,” making it easier to learn. The effect may also be described as an increase in focus, getting into the flow, or being in the zone.

The present technology employs hardware for NIEBS that attaches electrodes to the head of the patient. The hardware may also include speakers such as headphones. The present technology may apply NIEBS to a user and may or may not simultaneously play audio for the user via speakers such as headphones. The pulse for the NIEBS may or may not be based on the rhythm or beat of the audio signal. The speakers may or may not be combined into one frame or housing with the electrodes. Specifically, the speakers/headphones may be separate from the electrodes, or combined with the electrodes or may not be present.

The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

A NIEBS device may generate NIEBS signals that apply an appropriate electrical current to a user or patient, typically to the head. The type of electrical current used for NIEBS may vary and may or may not require supervision in a clinical setting. For example, a NIEBS treatment may combine both a type of electrical current that is typically considered safe and a type of current that may require clinical supervision such as direct current.

A healthcare professional such as a physician may prescribe a certain NIEBS treatment, or other electro medicine, to a patient. The certain NIEBS treatment may comprise a specified pattern of signals. The pattern may be related to the wave form of the signals, the intensity, length/duration of treatment, level of randomness or chaoticness of the treatment, the pattern of pulse trains in the treatment, etc. The present technology is to provide systems and methods for delivering the prescribed NIEBS treatment to the patient for use by the patient. The patient may use the NIEBS treatment in any location including at home or in a clinical setting. The prescription may require that the prescription be administered to the patient in a particular setting such as a clinical setting.

### Embodiments for Systems and Methods for Prescriptions for Noninvasive Electrical Brain Stimulation

### Prescription Management of NIEBS

The present technology may provide NIEBS or other electrotherapy to a user as prescribed by a healthcare professional. The NIEBS or other electro medicine prescription may provide for how the current pulse should be delivered (i.e. frequency, length of time, intensity, etc.). The prescription may be accomplished via a power tune. The power tune may be combined with any audio track or a specified audio track.

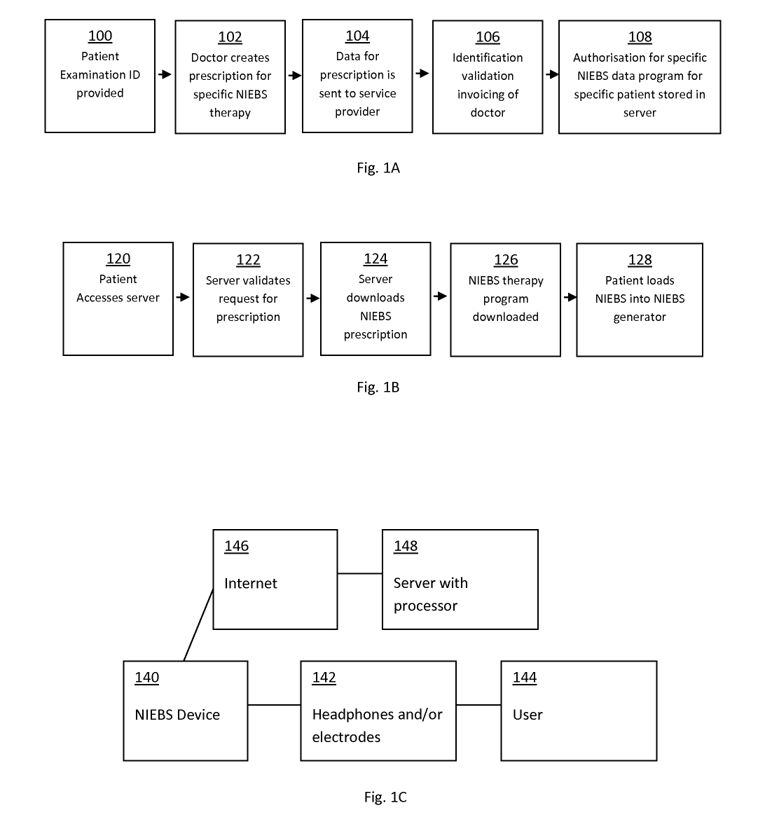

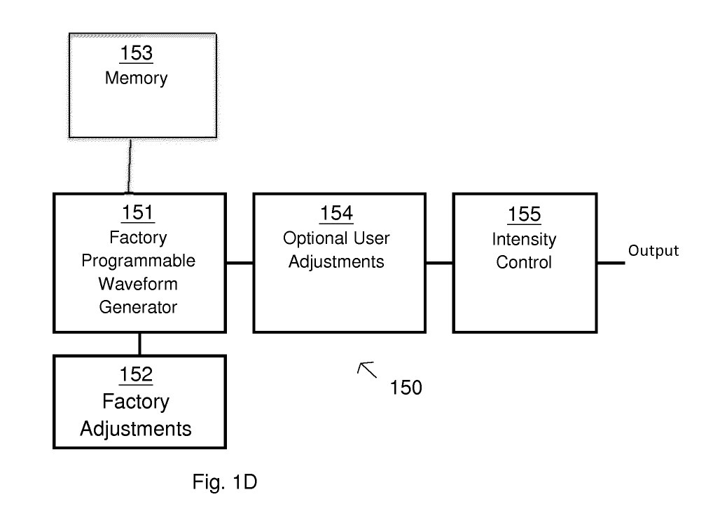

With reference to FIG. 1A which depicts the flow of how the prescriptions for NIEBS may be created and managed. FIG. 1A relates to the delivery of a therapy prescription to a dispensing agency. The management may involve a third party service provider.

First, the patient goes to visit the healthcare professional and is assigned an ID number such as a Patient Examination ID that may refer to the patient or a specific examination of the patient 100. The same Patient Examination ID may be reused for the same patient that comes back for reoccurring visits.

Second the healthcare professional performs an examination of the patient and creates a prescription 102 regarding a NIEBS treatment for the patient based on the examination.

Third, data for the prescription for the NIEBS treatment is sent by the healthcare professional to the third party service provider 104. The sending may be accomplished by the healthcare professional or someone associated with the healthcare professional such as a nurse or assistant. The prescription may also be entered by the healthcare professional into a computer device and then sent automatically by the computer device. For example the healthcare professional may use a handheld device such as a smartphone with an app for the present technology that automatically associates the Patient Examination ID and the prescription and sends such data to the third party service. The data for the prescription may be sent over a network such as the Internet.

Fourth, the third party service provider validates the data and/or identification of the healthcare professional 106. This validation may be described as billing validation invoicing. This validation process ensures that prescription is accurate for a given patient. However, validation may not be required in some embodiments.

Fifth, the third party service provider stores or maintains data for various NIEBS treatments. This may be accomplished via a database using servers associated with the third party service provider. Upon receiving the data for a prescription from the healthcare professional, the third party service provider authorizes a specific NIEBS data program based on the prescription for the patient associated with the Patient Examination ID 108.

With reference to FIG. 1B which depicts the flow of how the prescriptions for NIEBS may be managed and accessed by a patient. FIG. 1B relates to how a patient obtains a NIEBS therapy program via internet download.

First the patient accesses a server 120 such as the server of the third party server provider described in FIG. 1A. This may be done over the internet or other network. The patient may use a NIEBS device that has the ability to access networks and transfer data or may be done by a second device such as a computer, a notebook computer, or smart phone that is able to transfer the NIEBS prescription to the NIEBS device. For example, a smartphone may use a cellular connection or other data connection to access the network.

Second, the server associated with the third party service provider validates the request for the prescription 122. This may be accomplished via an ID number password or other means. The ID number may be the Patient Examination ID in FIG. 1A.

Third, the server transmits or sends the NIEBS prescription to the patient or makes the NIEBS prescription available to the patient for download 124.

Fourth, the patient downloads the NIEBS prescription 126. This may be accomplished over the internet. In one embodiment, the prescription is downloaded by the NIEBS device itself or an associated device such as a smartphone.

Fifth, the NIEBS program is loaded into the NIEBS generator of the device associated with patient used for NIEBS treatments 128. The NIEBS prescription may be a data file which contains data for how the NIEBS pulses should be delivered to the patient. In one embodiment, the patient uses the second device, such as a smartphone, to download the prescription and then uses the second device to transfer to prescription to the NIEBS device. For example, the NIEBS may have a physical data port or may use wireless communications such as Bluetooth.

It should be appreciated that FIGS. 1A and B may be computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of computer usable storage medium. In one embodiment, the methods may reside in a computer usable storage medium having instructions embodied therein that when executed cause a computer system to perform the method. In one embodiment, the NIEBS signals described herein are non-transitory but rather are sent over wired connections to the electrodes.

With reference to FIG. 1C which depicts an example environment and hardware for use with the methods described in FIGS. 1A and B and relates to a CES device. FIG. 1C depicts the server with a processor that may be associated with the third party service provider that receives NIEBS prescriptions from a healthcare professional and upon validation sends the prescription to the patient. The server may be associated with a database that stores a plurality of NIEBS data programs for use in NIEBS treatments and that may be prescribed as part of the prescriptions. The database may be a part of the server or may be remote to the server. The server may be connected to the Internet or other network. The example environment of FIG. 1C may also include headphones and/or electrodes 142, server with processor 148, internet 146, and NIEBS device 140,

The NIEBS device may be in communication with the server or may not have the abilities to communicate over the Internet and instead communicates with the second device. If the NIEBS device communicates with the server over the Internet then it may receive the NIEBS data programs from the server. The NIEBS data programs may be firmware for the NIEBS device. The NIEBS device may be capable of storing a plurality of NIEBS data programs and a particular NIEBS data program may be selected for use by the patient using controls associated with the NIEBS device or the NIEBS data program may be selected by controlling the NIEBS device via the second device.

In one embodiment, the second device retains the NIEBS data program and the NIEBS device is a dongle associated with the second device and is controlled via the second device. The second device may be a computer system, a handheld device, a smartphone, or other electronic device.

With reference to FIG. 1D which depicts an example environment and hardware for use with the methods described in FIGS. 1A and B. FIG. 1D depicts device 150 which may be the same as the NIEBS device of FIG. 1C. Device 150 has a programmable waveform generator that is able to generate NIEBS signals for use in a NIEBS treatment. For example, the NIEBS treatment may be output to electrodes worn by a patient. The Programmable waveform generator uses a NIEBS data program to generate the NIEBS treatment. The programmable waveform generator may be adjusted via factory adjustments at the time of manufacture. These factory adjustments may be overridden by subsequent NIEBS data programs. The programmable waveform generator may store NIEBS data programs in the memory block. The memory block may be hardwired to the programmable waveform generator or may be a removable memory such as an SD card or micro SD card. The optional user adjustments and Intensity Control may be controls that are operable by a user for controlling aspects of the NIEBS treatment via buttons or wheels. The controls may turn the treatment on or off, may control the amplitude or intensity of NIEBS signals, may control the duration of the treatment, etc. The example environment and hardware of FIG. 1D may include memory 153, factory programmable waveform generator 151, optional user adjustments 154, intensity control 155, factory adjustments 152, and an output.

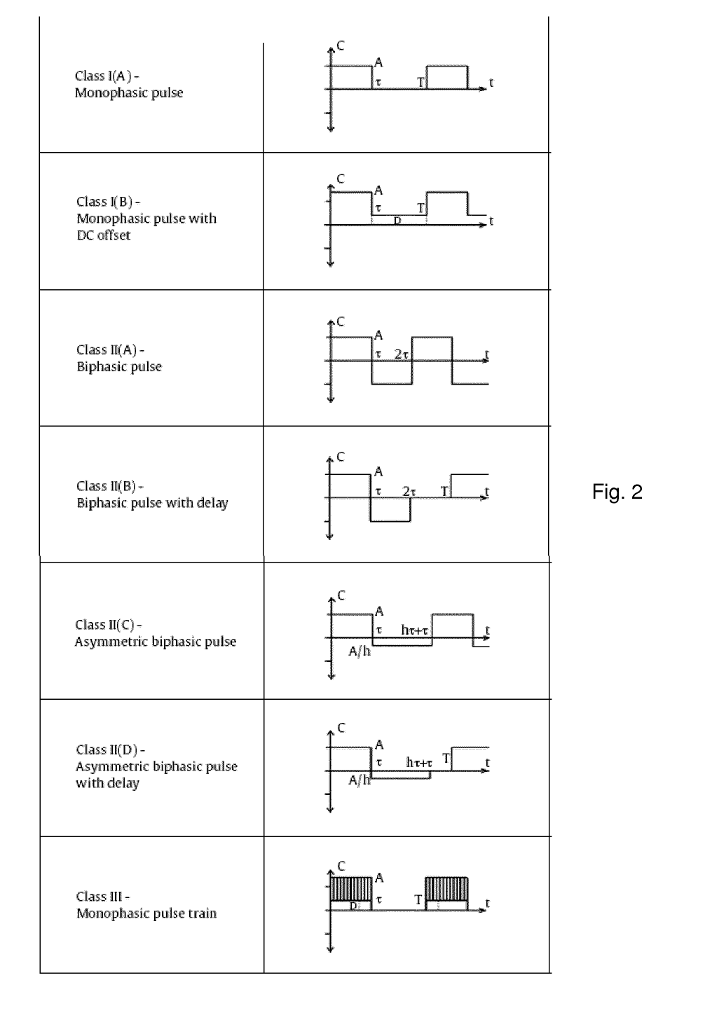

With reference to FIG. 2 which depicts wave forms that may be employed for use with the present technology. A NIEBS generator may receive wave forms from an audio source or from a waveform synthesizer associated with the NIEBS generator. The NIEBS generator may generate a NIEBS signal with associated wave forms for the NIEBS treatment. FIG. 2 depicts well known square wave forms for use in the present technology. The present technology is not limited to wave forms in FIG. 2 but may also employ other wave forms such as sine waves.

Wave forms for the present technology may be stored in a library and are used to create pulse patterns or pulse trains for use in NIEBS. The wave forms may be implemented via a programmable D/A converter. Research indicates that different pulse patterns have different effects on the brain, and that some pulse patterns have different effects on various conditions. Therefore, there is a need for a library of different pulse patterns to suit different health conditions.

The rate of pulses per second refers to a start of positive-going pulse to stop, with the delay until the next positive-going pulse starts. Like a sine wave, regardless of whether or not there is a negative-going pulse. “Beginning of a pulse rising, to the next time the pulse starts rising again.” The following are examples of pulse rates that may be employed by the present technology:

1. Pulse rate in range of 3-5 Hz. Low Freq.

2. Pulse rate in range of 50-100 Hz. Low Freq.

3. Pulse rate in range from 100-640 Hz. High Freq.

4. Pulse rate in range of 0.1-100 Hz

5. Direct current

Current level delivered: 1.5 mA. [milli-Ampere]

Current density on the skin: safety limit is between 25 and 60 microA/cm² [from Poreisz et al., 2007] The electric field across the brain tissue is on the order of less than 5 mV/mm, or 5 milli-Volts/millimeter.

Pulse pattern may be a Random Noise Stimulation pattern. Good results reported by Fertonani et al in paper “Random Noise Stimulation Improves Neuroplasticity in Perceptual Learning,” The Journal of Neuroscience, Oct. 26, 2011 31(43):15416-15423.

Noninvasive electrical brain stimulation (herein referred to as NIEBS) applies gentle micro-current pulses to the brain using electrodes. The electrodes of the present technology may be attached to a user's body at any number of locations. For example, for NIEBS, the electrodes are typically attached to the skin of the user's head and may be attached to the ears, earlobes, back of the skull, forehead, cheeks, etc. However, for both electrotherapy and NIEBS in general the electrodes may attached anywhere on the body such as to fingers, the arms, legs, torso, head, etc.

In NIEBS significant amounts of current pass the skull and reach cortical and subcortical structures. In addition, depending on the montage, induced currents at subcortical areas, such as midbrain, pons, thalamus and hypothalamus are of similar magnitude than that of cortical areas. Incremental variations of electrode position on the head surface also influence which cortical regions are modulated. The high-resolution modeling predictions suggest that details of electrode montage influence current flow through superficial and deep structures. Also, laptop based methods for tPCS dose design using dominant frequency and spherical models. These modeling predictions and tools are the first step to advance rational and optimized use of tPCS and NIEBS.

It is widely accepted that NIEBS stimulates the brain to manufacture neurotransmitters, like endorphins, which improve moods, emotions and cognitive capabilities. Noninvasive electrical brain stimulation has also been proposed for treatment following a stroke, brain trauma, high blood pressure, and Alzheimer's disease, as well as any or all neurological disorders, any or all mental disorders, and any or all cognitive enhancements. The present technology may also be used by healthy users or users who are not suffering from any diagnosed disorders or diseases. For example, a healthy user may be a student using the present technology to increase focus and learning abilities or may be an athlete using the present technology to increase sports performance.

The signals apparently normalize the electrical output of the brain. NIEBS has thus been used or tested to treat substance dependence, depression and anxiety. It has been noted in at least some instances that NIEBS has equal or greater efficacy for the treatment of depression when compared to antidepressant medications, with fewer side effects. NIEBS may be used specifically in combination with anti-depressant drugs and may be used to eliminate the side effects of central nervous system (CNS) medications or drugs in general. NIEBS may also be used in conjunction with other traditional medicine.

Treatments can be used in association with the present technology in ranges from less than one second up to an infinite number of seconds. The present technology is not limited to a particular range of duration, current, or frequency. The following ranges are meant as examples and do not limit the present technology. In one embodiment, a range is used from 10 to 30 minutes in duration although the treatments may extend up to 1½ hours depending on the electrical current configuration. The currents employed may be applied in pulse form or direct form with a pulse width in the range of from about 1 to about 500 milliseconds (ms) at a frequency of from about 0.1 Hertz (Hz) up to 1000 Hz with the current being less than 1 milliampere (mA) up to 5 mA

In accordance with an embodiment of the invention there is provided equipment for the implementation of a method as defined above, said equipment comprising a noninvasive electrical brain stimulation pulse generator and associated electrodes for applying pulses generated by the pulse generator to the head of a patient, WHEREIN the equipment includes multiple electrodes.

In an embodiment of the invention, there is an audio signal player and at least one associated loudspeaker for converting output from the signal player into audible sound. The at least one loudspeaker is preferably a pair of earphones and the noninvasive electrical brain stimulation pulse generator and sound signal generator may be built into a single unit, but are not necessarily thus combined.

Note that there are the following types of stimulation configurations:

1. Positive going pulse, with a direct current average in one direction. Class 1A and Class 1B deliver a varying amount of direct current in little bursts.

2. Alternating current pulses, where the direction of current alternates from positive going to negative going, as in Class IIA and Class IIB and IIC and IID. The average may be in one direction predominantly, or may average out to zero if the pulses are symmetric and equal in duration over time. You can see that for some modes, there is a net direct current passing thru the brain.

3. Class III shows a pulse train with a delay between delivery of a series of pulses.

The next paragraphs discuss how this delay may be configured, and is part of the overall therapy formulation that is available to a medical practitioner.

1. Random time period. Use a random number generator with a specified range in seconds. For example, 1-100 seconds. Run the random number generator which is set to produce a number between 1 and 100. Use that number as the time period between pulses. Run the generator after each pulse to determine the next time delay, or period, from the last pulse.

2. Semi-random time period.

Pick some time periods that are known to have some therapeutic effect. Make a table. For example:

| | | | | | | | | |
|---|---|---|---|---|---|---|---|---|
| Random No. | 1 | 3 | 5 | 10 | 20 | 40 | 60 | 100. |
| Bin containing | 1 | 2 | 3 | 4 | 5 | 6 | 7 | 8 |
| the delay | | | | | | | | |

Then randomly select from this group of time periods. Again, use a random number generator whose bounds are the number of allowed states. In the above example, there are 8 possible delay time periods. Set the random number generator to select any of the numbers from 1 to 8. Use the time delay associated with that bin number.

Say the random number generator picks 4. That means we use 10 second delay as the time period to the next pulse train initiation.

3. Periodic but increasing delay, with a plan

Here the time delay from one pulse train event to the next is arbitrarily set to predetermined sequence. It may be one with a set increase from one period duration to the next. As in 5 10 30 60 repeat 5 10 30 60.

4. Periodic, static period

Set delay to one of the group [1, 2, 3, 4, 5, 6, 7, 8, 9, 10] seconds. Or any other time period from 1 to 300 seconds, for example.

5. Continuous pulse train with no delay between any arbitrary group of pulses. Arbitrary duration of such pulse trains, selected from group [1-1000] seconds.

6. Direct Current Stimulation

No pulses, just application of a constant voltage for some time period. One could consider this a special case of a single positive going pulse with a really long time duration.

### Notes on Using Chaotic/Random Pulse for NIEBS:

Pulses or pulse trains for NIEBS and NIEBS prescriptions may be patterned or random. However, the idea of random pulses may not be desirable as random may still indicate a measureable structure impulse. The term chaotic pattern is better description of the pulse referred to herein. Chaotic may also be used to define the variety of the pauses or periods in between pulse trains.

### Computer Implemented Methods

It should be appreciated that the methods described herein may be computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of computer usable storage medium. In one embodiment, the methods may reside in a computer usable storage medium having instructions embodied therein that when executed cause a computer system to perform the method. In one embodiment, the NIEBS signals described herein are non-transitory but rather are sent over wired connections to the electrodes.

It is intended that the foregoing detailed description be regarded as illustrative rather than limiting and that it be understood that the detailed description should not be used to limit the scope of the invention.

1. A computer-implemented method for managing a noninvasive electrical brain stimulation “NIEBS” prescription, comprising:
receiving data for a prescription for a patient associated with a patient ID at a computer system, wherein said prescription is created by a healthcare professional after examining said patient;
validating said healthcare professional and said data for said prescription at said computer system;
maintaining a database or library of NIEBS data programs for NIEBS treatments at said computer system; and
authorizing a specific NIEBS data program for said patient at said computer system based on said prescription.

2. The method of claim 1, wherein the computer system is a third party service provider computer system.

3. The method of claim 1, wherein the data for the prescription may be sent to the computer system over a network.

4. The method of claim 1, wherein the prescription may be sent to the computer system using a handheld device.

5. A computer-implemented method for managing a noninvasive electrical brain stimulation “NIEBS” prescription, comprising:
sending a request from a device to a server for a NIEBS prescription;
receiving said NIEBS prescription from said server, wherein a patient identity associated with said prescription is validated by said server.

6. The method of claim 5, wherein the device is a patient NIEBS device.

7. The method as recited in claim 5, further comprising:
loading said prescription into a NIEBS generator associated with said patient NIEBS device; and
generating NIEBS signals for NIEBS treatment at said patient NIEBS device based on said prescription.

8. The method of claim 5, further comprising
transferring said prescription from said device to a patient NIEBS device associated with the device.

9. The method as recited in claim 8, further comprising:
loading said prescription into a NIEBS generator associated with said patient NIEBS device; and
generating NIEBS signals for NIEBS treatment at said patient NIEBS device based on said prescription.

10. The method as recited in claim 8 wherein said device is a smart phone, a hand held device, or a computer system and communicates with said patient NIEBS device via at least one of a network connection, a Bluetooth connection, a wired connection, a removable memory card, a near field communication, and Wi-Fi.

11. The method as recited in claim 8 wherein said patient NIEBS device is a dongle that is powered by an audio source associated with said second device.

12. A computer-implemented method for delivering a noninvasive electrical brain stimulation “NIEBS” prescription, comprising:
receiving a request from a patient device at a server for a NIEBS prescription;
validating a patient identity associated with said prescription;
upon validation, sending said NIEBS prescription from said server to said patient device to be used by a patient associated with said patient identity for NIEBS treatment.

13. A computer-implemented method for delivering a specific noninvasive electrical brain stimulation “NIEBS” signal formulation for a patient, comprising:
providing a prescription for a specific NIEBS signal formulation by a medical professional;
forwarding said prescription to a server operated by a service provider of NIEBS services;
providing a payment verification mechanism for verifying payment for said prescribed services; and
performing authorization for delivery of said NIEBS signal formulation in a computer-readable format by download from the server.

14. The method of claim 13 wherein said computer-readable format is obtained from a library of NIEBS signal formulations.

15. The method of claim 13 wherein said computer-readable format is generated by a manual process, according to specifications in the prescription from the medical professional.

16. The method of claim 15 wherein the manual process invokes the use of a software toolkit designed for assembling NIEBS formulations.

17. (canceled)

18. A computer-readable medium comprising computer-readable instructions to implement the method of claim 1.

19. An apparatus configured to perform the method of claim 1.

20. An apparatus for delivering a specific NIEBS signal formulation for a patient, comprising:
a prescription providing mechanism for providing a prescription for a specific NIEBS signal formulation created by a medical professional;
a delivery mechanism for forwarding said prescription to a server operated by a service provider of NIEBS services;
a payment verification mechanism for verifying payment for said prescribed services; and
an authorization mechanism for providing authorization for delivery of said NIEBS signal formulation in a computer-readable format by download from the server.

21-33. (canceled)

34. An apparatus for delivering a specific NIEBS signal formulation for a patient, comprising:
a prescription for a specific NIEBS signal formulation created by a medical professional;
a delivery mechanism for forwarding said prescription to a server operated by a service provider of NIEBS services;
a payment verification mechanism for said prescribed services; and
authorization for delivery of said NIEBS signal formulation in a computer-readable format by download from the server.
